(19) United States
(12) Patent Application Publication
(10) Pub. No.: US 2020/0257270 A1
(43) Pub. Date: Aug. 13, 2020

(54) MASTER-SLAVE SYSTEM AND CONTROL METHOD OF THE SAME

(71) Applicant: Sony Corporation, Tokyo (JP)

(72) Inventors: Hiroyuki SUZUKI, Tokyo (JP); William Alexandre CONUS, Tokyo (JP); Ryuta HORIE, Saitama (JP)

(73) Assignee: Sony Corporation, Tokyo (JP)

(21) Appl. No.: 16/758,411

(22) PCT Filed: Sep. 7, 2018
(86) PCT No.: PCT/JP2018/033137

§ 371 Date: Apr. 23, 2020

(30) Foreign Application Priority Data
Oct. 30, 2017 (JP) 2017-208806

(51) Int. Cl. G05B 19/4155; A61B 17/29; A61B 34/37; A61B 17/295; A61B 90/00

(52) U.S. Cl. CPC G05B 19/4155; A61B 17/29; A61B 34/37; A61B 2017/00084; A61B 90/06; G05B 2219/49216; A61B 17/295

(57) ABSTRACT

Provided are a master-slave system where a master apparatus remotely operates a slave apparatus and a control method of this system.

The master-slave system includes a slave apparatus that includes a temperature acquisition unit that acquires a temperature and a master apparatus that includes a presentation unit that presents the acquired temperature. The presentation unit includes a temperature change unit that is provided on the operation unit and produces a temperature change on the basis of the acquired temperature to produce a temperature change in harmony with the acquired temperature. Moreover, the master-slave system further includes a determination unit that determines a type or a characteristic of the operation target on the basis of an input temperature input by the temperature input unit and the temperature acquired by the temperature acquisition unit.

### TECHNICAL FIELD

A technology disclosed in the present description relates to a master-slave system where a master apparatus remotely operates a slave apparatus, and a control method of this mater-slave system.

### BACKGROUND ART

Robotics technologies are making remarkable progress in recent years and are increasingly widespread in worksites of various industrial fields. A master-slave type robot system is used in an industrial field where complete autonomous operation under computerized control is still difficult, such as a medical field. For example, a master-slave type medical robot is used in an endoscopic surgery such as a laparoscopic surgery and a thoracoscopic surgery. In such case, a surgeon can remotely operate a slave arm which has an end effector to which a surgery tool such as forceps is attached to perform the surgery while viewing a surgical field on a 3D monitoring screen.

Considering invasion into a biological tissue or surgical efficiency in using an endoscope, it is preferable to present information received by the end effector of the slave from an affected site or the like to a user on the master side, similarly to a sense of touch obtained by an operating surgeon touching the affected site during a surgery not using a robot. For example, as a type of medical master-slave type robot systems, there has also been proposed a master-slave type surgical system which detects an external force acting on an end effector such as a gripped portion (gripper), and presents a force sense to a surgeon (for example, see PTL 1).

### SUMMARY

### Technical Problem

According to the conventional technology, however, only a force sense is presented to the surgeon, and a temperature sense (temperature), which is one of original senses of touch obtained when the surgeon touches an affected site, is difficult to present. Accordingly, an object of the technology disclosed in the present description is to provide a master-slave system capable of presenting a temperature and a control method of this master-slave system.

### Solution to Problem

A first aspect of the technology disclosed in the present description is a master-slave system including

a slave apparatus that includes a temperature acquisition unit that acquires a temperature, and

a master apparatus that includes a presentation unit that presents the acquired temperature.

Note that the term “system” used herein refers to a logical aggregation of a plurality of apparatuses (or function modules implementing specific functions). It does not matter whether or not the respective apparatuses or function modules are contained in a single housing.

For example, the temperature acquisition unit includes a temperature sensor attached to an end effector of the slave apparatus.

In addition, the presentation unit presents a temperature sense corresponding to the temperature acquired by the temperature acquisition unit. The presentation unit may include a display unit that displays information associated with the temperature acquired by the temperature acquisition unit.

The master-slave system may further include a determination unit that determines a state of an operation target treated by the end effector, on the basis of the temperature acquired by the temperature acquisition unit. Moreover, the master-slave system may further include a force sensor that detects an external force acting on the end effector. The determination unit may determine the state of the operation target on the basis of a combination of a force sense obtained by the force sensor and information indicating the temperature acquired by the temperature acquisition unit.

Furthermore, the master apparatus may include an operation unit used to operate the slave apparatus. The presentation unit may present the temperature sense based on the acquired temperature, by the operation unit. Accordingly, the presentation unit may include a temperature change unit that is provided on the operation unit and produces a temperature change on the basis of the acquired temperature. The temperature change unit produces the temperature change in harmony with the acquired temperature. For example, the temperature change unit produces the temperature change while controlling a speed or acceleration of a temperature change of the temperature acquired by the temperature acquisition unit.

In addition, a second aspect of the technology disclosed in the present description is a control method of a master-slave system, the control method including

a temperature acquisition step that causes a slave apparatus to acquire a temperature, and

a presentation step that causes a master apparatus to present the temperature acquired by the slave apparatus.

### Advantageous Effect of Invention

According to the technology disclosed in the present description, a master-slave system capable of presenting a temperature and a control method of this master-slave system can be provided.

Note that the advantageous effect described in the present description is presented only by way of example. Advantageous effects of the present invention are not limited to such advantageous effect. In addition, the present invention may also offer additional advantageous as well as the advantageous effect described above.

Further objects, characteristics, and advantages of the technology disclosed in the present description will be clarified through more detailed description based on an embodiment described below and accompanying drawings.

### BRIEF DESCRIPTION OF DRAWINGS

FIG. 1 is a diagram schematically depicting a functional configuration example of a master-slave system 1 of a master-slave type.

FIG. 2 is a diagram schematically depicting a configuration example of an end effector and a support arm apparatus.

FIG. 3 is an enlarged diagram depicting an end effector 200.

FIG. 4 is a diagram depicting a configuration example of a first blade 201 including a flexural element.

FIG. 5 is a diagram for explaining a method for providing a flexure detection element using an FBG sensor on the first blade 201.

FIG. 6 is a diagram for explaining the method for providing the flexure detection element using the FBG sensor on the first blade 201.

FIG. 7 is a diagram depicting a modification of the first blade 201.

FIG. 8 is a diagram depicting a state where a temperature sense presentation unit is disposed on a grip 800 used as an input unit 11.

FIG. 9 is a diagram depicting a specific configuration example of a temperature sense feedback function unit 50.

FIG. 10 is a diagram depicting an example of a relationship between a temperature T detected by a temperature sensor 51 and a target temperature T_{ref} instructed by a temperature sense presentation unit 53.

FIG. 11 is a diagram depicting an example of the relationship between the temperature T detected by the temperature sensor 51 and the target temperature T_{ref} instructed by the temperature sense presentation unit 53.

FIG. 12 is a diagram depicting a configuration example of the end effector (forceps) 200 which includes a temperature sensor provided on the first blade 201 and a temperature input unit 54 provided on a second blade 202.

FIG. 13 is a diagram depicting a hardware configuration of an information processing apparatus 2100.

### DESCRIPTION OF EMBODIMENT

An embodiment of the technology disclosed in the present description will hereinafter be described in detail with reference to the drawings.

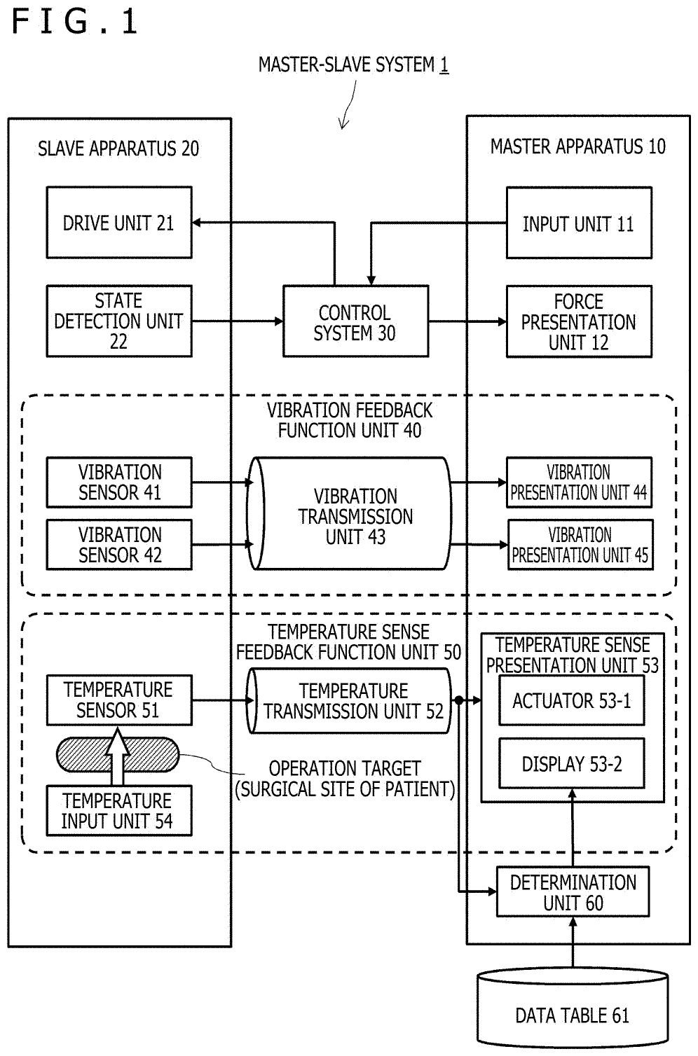

FIG. 1 schematically depicts a functional configuration example of a master-slave system 1 of a master-slave type to which the technology disclosed in the present description is applicable. For example, the master-slave system 1 depicted in the figure is a medical robot system which performs endoscopic surgeries such as a laparoscopic surgery and a thoracoscopic surgery. A support arm apparatus (not depicted in FIG. 1) on the slave side and an end effector (a medical tool such as surgical forceps) attached to this support arm apparatus are driven according to an instruction input from a user on the master side via an input apparatus such as a controller to perform various treatments for a surgical site of a patient using this end effector.

The master-slave system 1 depicted in the figure includes a master apparatus 10, a slave apparatus 20, and a control system 30 which drives the slave apparatus 20 according to an instruction input from the user via the master apparatus 10. When the user operates the master apparatus 10, the slave apparatus 20 is remotely operated according to an operation command transmitted to the slave apparatus 20 via the control system 30 using wired or wireless communication means.

The master apparatus 10 includes an input unit 11 through which the user such as a surgeon performs an input operation and a force presentation unit 12 which presents a force to the user operating the input unit 11.

For example, the input unit 11 may include an input mechanism such as a lever, a grip, a button, a jog dial, a tact switch, and a foot pedal switch. However, a specific configuration of the input unit 11 is not limited to the configuration described above. Various known configurations allowed to be included in an input apparatus of an ordinary master-slave type robot system are available.

In addition, for example, the force presentation unit 12 includes a servo motor which drives the lever, the grip, and the like constituting the input unit 11 and further includes a servo motor which drives the support arm apparatus (not depicted) supporting the input unit 11, and others. The force presentation unit 12 presents to the user, as a force sense (also called haptic feedback), a force acting on a medical tool on the slave apparatus 20 side by driving the lever, the grip, and the like constituting the input unit 11 in such a manner as to give a resistance to an operation input from the user through the input unit 11, for example, according to the force acting on the medical tool.

On the other hand, the slave apparatus 20 includes the support arm apparatus having a tip to which a medical surgery tool such as forceps as an end effector is attached, a drive unit 21 which drives this support arm apparatus and the medical surgery tool disposed at the tip, and a state detection unit 22 which detects a state of the support arm apparatus. Note that a medical surgery tool touching a patient during a surgical operation, such as tweezers and a cutting tool as well as forceps, a catheter system, and an imaging apparatus such as an endoscope and a microscope may be attached to the tip of the support arm apparatus.

For convenience, FIG. 1 does not depict the end effector and the support arm apparatus supporting the end effector. The support arm apparatus has six-degree-of-freedom position and posture for changing a position and a direction of the end effector attached to a tip (or distal end) of the support arm apparatus in a three-dimensional space. Moreover, in a case where the end effector has a gripping mechanism such as forceps, the support arm apparatus has additional one degree of freedom for gripping an object using forceps.

For example, the drive unit 21 corresponds to an actuator unit for driving a link mechanism constituting the support arm apparatus. Moreover, in a case where the end effector of the support arm apparatus is a medical tool having a drive portion such as forceps, the drive unit 21 also corresponds to an actuator for operating this drive portion of the end effector (e.g., an actuator for opening and closing forceps). By driving a corresponding motor according to a control amount calculated by the control system 30, an arm unit and the end effector (a medical tool such as forceps) operate in accordance with an instruction given from the user via the master apparatus 10.

For example, the state detection unit 22 includes a force sensor (torque sensor) which detects an external force acting on respective links and the end effector of the support arm apparatus, an encoder which detects rotation angles of joints of the support arm apparatus, and the like.

The control system 30 implements, between the master apparatus 10 and the slave apparatus 20, transmission of information associated with drive control of the support arm apparatus on the slave apparatus 20 side and transmission of information associated with force presentation from the slave apparatus 20 to the master apparatus 10 side. However, some or all of functions of the control system 30 may be equipped at least on either the slave apparatus 20 or the master apparatus 10. For example, a CPU (Central Processing Unit) (not depicted) of at least either the master apparatus 10 or the slave apparatus 20 functions as the control system 30. Alternatively, respective CPUs of the master apparatus 10 and the slave apparatus 20 function as the control system 30 in cooperation with each other. A specific configuration of the control system 30 will be described below.

The master-slave system 1 according to the present embodiment further includes a vibration feedback function unit 40 and a temperature sense feedback function unit 50. The vibration feedback function unit 40 feeds back a vibration generated in the end effector (a medical tool such as forceps) on the slave apparatus 20 side to the user operating the master apparatus 10. In addition, the temperature sense feedback function unit 50 feeds back information indicating a temperature or a temperature change of a target (a surgical site of a patient) treated by the end effector to the user operating the master apparatus 10. By the vibration feedback function unit 40 and the temperature sense feedback function unit 50 included in the master-slave system 1, the user is given a haptic sense and a temperature sense in addition to a force sense during an endoscopic surgery using the master apparatus 10. Accordingly, the user can easily obtain a sense of touch with respect to a body tissue of the patient treated on the slave apparatus 10 side.

The vibration feedback function unit 40 includes a first vibration sensor 41 and a second vibration sensor 42 that are attached near the end effector on the slave apparatus 20 side, a vibration transmission unit 43 which transmits detection signals generated by the vibration sensors 41 and 42, and a first vibration presentation unit 44 and a second vibration presentation unit 45 that are attached to the input unit 11 or the like of the master apparatus 10.

The first vibration sensor 41 includes a condenser microphone, for example, and detects an auditory vibration (i.e., sound) generated in the end effector such as forceps. On the other hand, the second vibration sensor 42 includes an accelerometer, for example, and detects a haptic vibration generated in the end effector.

The vibration transmission unit 43 performs amplification processing for respective detection signals of the first vibration sensor 41 and the second vibration sensor 42, appropriately performs signal processing, such as filtering processing and waveform equalization processing for extracting only components of a significant frequency band, for the detection signals, and thereafter respectively transmits the detection signals to the first vibration presentation unit 44 and the second vibration presentation unit 45.

The first vibration presentation unit 44 includes audio output equipment such as a speaker and an earphone, for example, and gives an auditory vibration corresponding to a detection signal of the first vibration sensor 41 to the user operating the input unit 11 of the master apparatus 10. On the other hand, the second vibration presentation unit 45 includes a vibration element such as a voice coil, for example, and gives a haptic vibration corresponding to a detection signal of the second vibration sensor 42 to the user operating the input unit 11.

The temperature sense feedback function unit 50 includes a temperature sensor 51 attached to the end effector on the slave apparatus 20 side, a temperature transmission unit 52 which transmits a detection signal generated by the temperature sensor 51, and a temperature sense presentation unit 53 which presents information indicating a temperature transmitted to the user operating the master apparatus 10.

The temperature sensor 51 measures a surface temperature of an operation target in contact with the end effector. The temperature sensor 51 includes a flexure sensor capable of detecting a flexure produced in the end effector due to a temperature change, for example, and is capable of calibrating a flexure amount produced in the end effector into a temperature change using a calibration matrix. Note that details of a configuration of the temperature sensor 51 will be described below. The temperature transmission unit 52 transmits a detection signal generated by the temperature sensor 51 on the slave apparatus 20 side to the master apparatus 10.

The temperature sense presentation unit 53 is attached to the input unit 11 of the master apparatus 10, for example, and presents a temperature sense corresponding to a temperature detected by the temperature sensor 51 (sense generated by the temperature) to the user operating the input unit 11. For example, the input unit 11 may include an input device such as a lever, a grip, a button, a jog dial, a tact switch, and a foot pedal switch. The temperature sense presentation unit 53 is disposed on the above input device at a location where the input device comes into contact with a skin of a fingertip or the like of the user to give the user a temperature sense corresponding to a temperature change of the end effector measured by the temperature sensor 51. In such manner, a sense of touch with respect to an operation target in contact with the end effector can effectively be reproduced.

The temperature sense presentation unit 53 includes a temperature actuator 53-1 capable of freely changing a temperature in at least a positive direction or a negative direction at least within a predetermined temperature range. The temperature actuator 53-1 is disposed on the master apparatus 10 at a location where the master apparatus 10 comes into contact with a body of the user, such as the input unit 11, and presents a temperature sense by changing a temperature on the basis of a temperature change detected by the temperature sensor 51 (or transmitted via the temperature transmission unit 52), for example.

The temperature actuator 53-1 basically produces a temperature change in harmony with a temperature detected by the temperature sensor 51 (or transmitted via the temperature transmission unit 52). More specifically, the temperature of the temperature actuator 53-1 increases according to an increase in a surface temperature of an operation target in contact with the end effector on the slave apparatus 20 side. Conversely, the temperature of the temperature actuator 53-1 decreases according to a decrease in the surface temperature of the operation target.

In such case, the temperature actuator 53-1 achieves a temperature change while amplifying the temperature change detected by the temperature sensor 51. Accordingly, the user can easily sense a change of the surface temperature of the operation target and obtain a higher temperature sense. Alternatively, the temperature actuator 53-1 also achieves a temperature change while damping the temperature change detected by the temperature sensor 51. Accordingly, presentation of a temperature to the user without invasion is achievable.

Furthermore, the temperature actuator 53-1 may present a temperature to the user while controlling a speed or acceleration of the temperature change detected by the temperature sensor 51 to control sensitivity of the user for detecting the temperature.

For example, the temperature actuator 53-1 may include a Peltier element or a micropump which conducts a temperature using fluid or gas. The temperature actuator 53-1 preferably has high responsiveness and transmissibility of both temperatures lower and higher than room temperature. From such viewpoint, it is considered that the temperature sense presentation unit 53 preferably includes a micropump.

In addition, the temperature sense presentation unit 53 includes a display 53-2. The display 53-2 may be provided on the master apparatus 10 at a location observable by the user and may display information associated with a temperature detected by the temperature sensor 51 on a screen. For example, the temperature sense presentation unit 53 may display a numerical value or a color corresponding to a temperature detected by the temperature sensor 51 on the display 53-2.

Moreover, the master-slave system 1 may further include a determination unit 60 added to the temperature sense feedback function unit 50. The determination unit 60 determines a state of an operation target in contact with the end effector, on the basis of a temperature detected by the temperature sensor 51 (or transmitted via the temperature transmission unit 52).

For example, when blood comes into contact with the temperature sensor 51, the determination unit 60 can determine that a surgical site of the patient corresponding to an operation target is bleeding, on the basis of a temperature difference between an environmental temperature and the blood. In addition, when the temperature sensor 51 touches an object that is present at an invisible place, the determination unit 60 can determine which tissue is in contact (e.g., which of fat and a blood vessel is in contact), on the basis of a temperature difference. The determination unit 60 may be configured to perform such determination process with reference to a data table 61 which describes a correspondence between a temperature and a state of a body tissue of the patient.

Moreover, the determination unit 60 can determine whether or not an operation target (a body tissue of the patient) in contact with the end effector is a normal tissue on the basis of a combination of a temperature detected by the temperature sensor 51 and information indicating an external force (i.e., force sense) that is acting on the end effector and is detected by the state detection unit 22. The determination unit 60 may be configured to perform such determination process with reference to the data table 61 which describes a correspondence between a temperature and a force sense and a state of a body tissue of the patient.

In addition, a determination result obtained by the determination unit 60 may be displayed on the screen of the display 53-2 of the temperature sense presentation unit 53. According to the system configuration example depicted in FIG. 1, the determination unit 60 is disposed on the master apparatus 10 side. In a modification, the determination unit 60 may be provided not on the master apparatus 10 but on the slave apparatus 20 side. In such case, the determination result obtained by the determination unit 60 may be transmitted from the slave apparatus 20 to the master apparatus 10 side.

Here, in a case where the end effector includes a device which has two or more contact portions in contact with an operation target, such as forceps for pinching an operation target, the temperature sensor 51 may be provided on one of the contact portions. In such case, further, the temperature input unit 54 may be provided on another contact portion. The temperature input unit 54 includes a temperature actuator, such as a heating element, capable of freely changing a temperature at least within a predetermined temperature range.

In such case, a type or a characteristic of an operation target can be determined on the basis of a relationship between an input temperature input to an operation target by the temperature input unit 54 and a temperature of the operation target measured by the temperature sensor 51. The determination unit 60 described above may further perform such determination process. Alternatively, another determination unit (not depicted) which performs a determination process for determining a type or a characteristic of an operation target may additionally be provided. In addition, such determination process may be performed with reference to the data table 61 which describes a correspondence between a transmission function of an input/output temperature characteristic and the type or the characteristic of the operation target (e.g., a body tissue of the patient).

Note that FIG. 1 depicts only a configuration particularly necessary for describing the embodiment according to the technology disclosed in the present description. The master-slave system 1 may have other function blocks included in an ordinary master-slave type robot system as well as the function blocks depicted in the figure. Various known configurations are applicable to configurations not depicted in the figure. Accordingly, detailed description of these configurations is omitted in the present description.

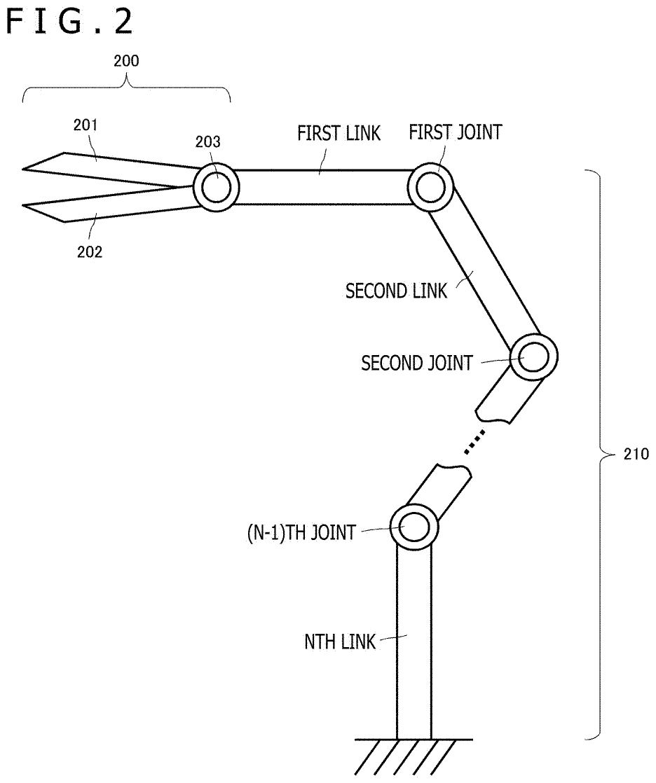

The temperature sensor 51 equipped on the slave apparatus 20 will next be described in detail. FIG. 2 schematically depicts a configuration example of an end effector 200 and a support arm apparatus 210 usable on the slave apparatus 20 side.

The support arm apparatus 210 is assumed to have such a configuration that the end effector 200 is attached at a distal end and that a proximal end is connected to the drive unit 21 (described above) of the slave apparatus 20. According to the example depicted in the figure, the support arm apparatus 210 includes a multijoint arm and has six-degree-of-freedom position and posture to change a position and a direction of the end effector 200 in a three-dimensional space.

For convenience, respective links included in the multijoint arm of the support arm apparatus 210 are referred to as a first link, a second link, and so on in order starting from the distal end side (or a rear end of the end effector 200). In addition, respective joints included in the multijoint arm are referred to as a first joint, a second joint, and so on in order starting from the distal end side. Configurations of the multijoint arm, such as degree-of-freedom configurations of the number of axes (or the number of joints) and the respective axes, and the number of links (or the number of arms) may be any kind. Additionally, the support arm apparatus 210 is not required to have a specific multijoint arm structure in implementing the technology disclosed in the present description. For example, a parallel link apparatus combining a plurality of link mechanisms in parallel (for example, see PTL 2) is applicable to the support arm apparatus 210.

The end effector 200 is a surgical tool constituting forceps which include a pair of a first blade 201 and a second blade 202 and a forceps rotation shaft 203 which connects the pair of blades such that the blades are rotatable relative to each other. The end effector 200 opens and closes according to rotations of the first blade 201 and the second blade 202 in opposite directions around the forceps rotation shaft 203 to pinch, push open, and press an operation target such as a body tissue and a surgical tool. The first blade 201 and the second blade 202 are connectable to each other in such a manner as to be openable and closable by using an appropriate gear mechanism to constitute the forceps rotation shaft 203, for example. However, a structure itself of the end effector 200 does not directly relate to the technology disclosed in the present description. Accordingly, detailed description of this structure is omitted.

While not depicted in FIG. 2, the temperature sensor 51 which measures a surface temperature of an operation target in contact with the end effector 200 is attached to the end effector 200, as already described above. Various types of sensor elements are adoptable as the temperature sensor 51. According to the present embodiment, however, a flexure sensor capable of detecting a flexure produced in the forceps 200 due to a temperature change is employed.

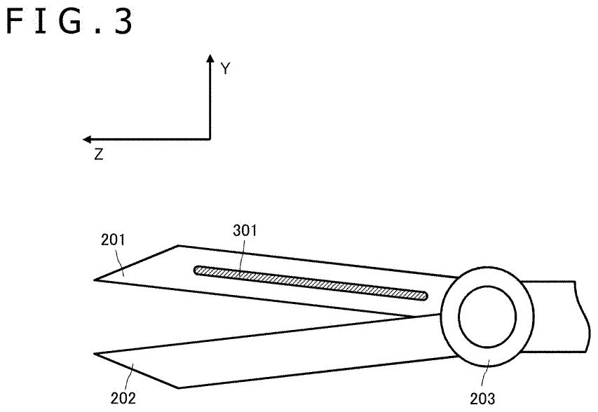

FIG. 3 is an enlarged diagram of the forceps 200 provided as the end effector. Note that an XYZ coordinate system which defines a major axis of the end effector 200 as a Z-axis is established. Accordingly, the left direction in the sheet surface corresponds to the Z-axis, a direction perpendicular to the sheet surface corresponds to an X-axis, and an up-down direction in the sheet surface corresponds to a Y-axis.

A flexure detection element 301 for detecting a flexure of the first blade 201 is attached to the first blade 201. The first blade 201 can be considered as a cantilever which has the forceps rotation shaft 203 as a fixed end. In addition, when the forceps 200 pinch an operation target (a body tissue of the patient or the like), for example, the first blade 201 flexes at least in one of the X, Y, or Z direction according to a temperature change produced by contact with the operation target. A detection signal generated by the flexure detection element 301 can be calibrated into a temperature change, using a calibration matrix which performs calibration into a temperature change.

From a viewpoint of measuring a flexure of the first blade 201, it is more preferable that the first blade 201 is a flexural element which has an easily flexible shape instead of a simple blade shape. For example, when a hole or a notch is formed in a simple blade shape, the blade shape easily flexes according to thermal expansion or the like. Accordingly, performance as a flexural element improves.

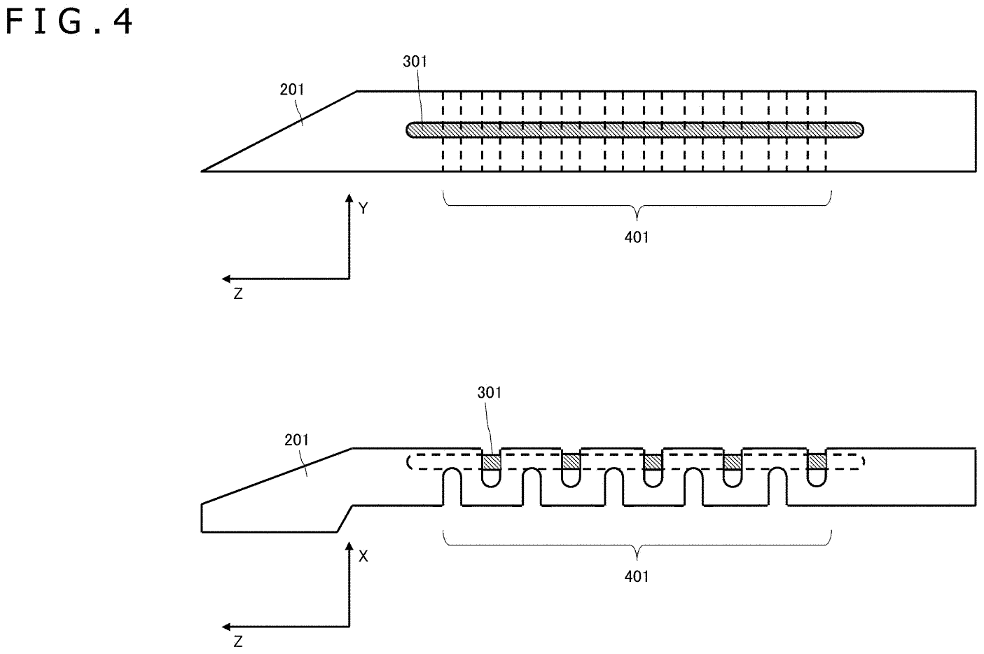

A specific configuration example of the first blade 201 including a flexural element will be described with reference to FIG. 4. This figure depicts a side surface (Y-Z surface) and an X-Z cross section of the first blade 201 a part of which includes a flexural element 401. The flexure detection element 301 is attached to the Y-Z surface. The flexural element 401 having a meandered structure is formed in a part of the first blade 201. The flexural element 401 which has a meander structure including repeated folds or meandering is present in the Z-X plane as depicted in the figure. Accordingly, the first blade 201 easily compresses and expands in the Z direction and the X direction according to a temperature change produced at the time of contact with a surface of a heat source (a body tissue of the patient or the like). In other words, a structure of the flexural element 401 is formed at least in a part of the first blade 201.

A flexure produced in the first blade 201 due to a temperature change is easily detectable by attaching the flexure detection element 301 to the portion of the flexural element 401 in the first blade 201 as depicted in FIG. 4. However, the flexural element 401 constituted on the first blade 201 is not particularly limited to have the meander structure and may have other various shapes available as a flexural element easily flexible according to a temperature change.

For example, each of the first blade 201 and the second blade 202 is manufactured using stainless steel (Steel Use Stainless: SUS), a Co—Cr alloy, or a titanium material each known as a metal material having excellent biocompatibility. From a viewpoint that the flexural element 401 is formed on a part of the structure of the first blade 201 as described above, it is preferable that the first blade 201 is manufactured using a material having such mechanical characteristics as high strength and low rigidity (low Young's modulus), such as a titanium alloy.

In short, the end effector 200 as a long and narrow tubular component has such a configuration where at least one flexural element and at least one flexure detection element are disposed between the distal end and the proximal end and is capable of measuring a flexure of the end effector 200 produced by a temperature change at the time of contact with an operation target such as a body tissue of the patient.

The flexure detection element 301 may be a capacitance sensor, a semiconductor flexure gauge, a foil flexure gauge, or the like widely known in the corresponding industry. According to the present embodiment, however, an FBG (Fiber Bragg Grating) sensor manufactured using optical fibers is adopted.

Here, (as is known), the FBG sensor is a sensor, which includes diffraction gratings (gratings) formed along a major axis of optical fibers, and is capable of detecting, as a wavelength change of reflection light with respect to that change of incident light having a predetermined wavelength band (Bragg wavelength), a change of intervals of the diffraction gratings resulting from expansion or compression according to a flexure or a temperature change produced by an acting force. In addition, the wavelength change detected by the FBG sensor can be converted into a flexure, a stress, or a temperature change causing the wavelength change. The FBG sensor using optical fibers can maintain high detection accuracy under an assumed use environment from a viewpoint of an in-house heating and a low transmission loss produced by wiring (difficulty in generating noise from the outside). Moreover, the FBG sensor is advantageous in easy handling for sterilization necessary for medical treatments or under a strong magnetic field.

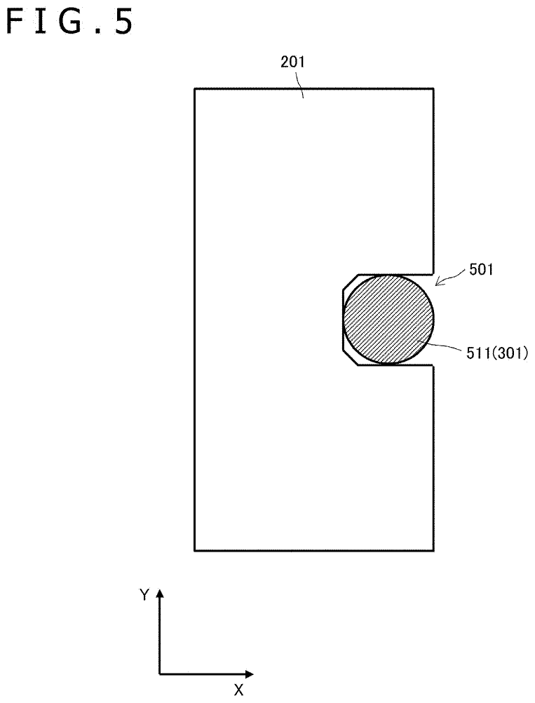

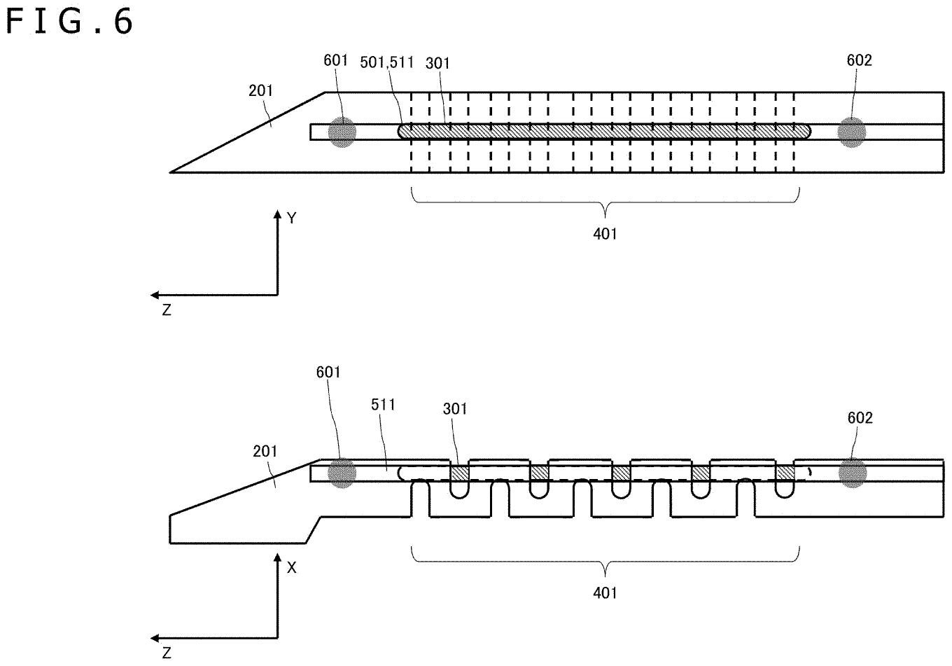

Described with reference to FIGS. 5 and 6 is a method for equipping the flexure detection element 301, which uses the FBG sensor, on the first blade 201.

FIG. 5 depicts an X-Y cross section of the first blade 201. One groove 501 is formed in a surface of the first blade 201 in the major axis direction (Z direction). In addition, respective optical fibers 511 are embedded in the grove 501 to be attached to the first blade 201 such that a contour of the first blade 111 is not enlarged. The optical fibers 511 are fixed to the surface of the first blade 201 at several positions (described below) by an adhesive or the like. The first blade 201 flexes according to a temperature change produced at the time of contact with an operation target (a body tissue or the like) corresponding to a heat source. At this time, the optical fibers 511 flex together with the first blade 201 as one piece body.

A portion where diffraction gratings are formed in the optical fibers 511 attached to the first blade 201 operates as the FBG sensor. Accordingly, diffraction gratings are formed in a range that is overlapping with the flexural element 401 (described above) and is included in the optical fibers 511 provided in the major axis direction of the first blade 201 to constitute the FBG sensor used as the flexure detection element 301 which detects a flexure of the first blade 201 produced by a temperature change.

Moreover, FIG. 6 depicts the side surface (Y-Z surface) where the groove 501 described above is formed, and the X-Z cross section, each included in the first blade 201. The optical fibers 511 are embedded in the one groove 501 formed in the surface of the first blade 201 in the major axis direction (Z direction). A range included in the optical fibers 511 and overlapping with the flexural element 401 is a portion where diffraction gratings are formed to constitute the FBG sensor and is used as the flexure detection element 301. The portion where the FBG sensor is provided in the optical fibers 511 is filled with diagonal lines in FIG. 6.

In addition, the optical fibers 511 are fixed by an adhesive or the like to both of ends 601 and 602 of a portion where the FBG sensor (i.e., flexure detection element 301) is provided, in the surface of the first blade 201. Accordingly, when a part of the first blade 201 corresponding to the flexural element 401 flexes by contact with an operation target (a body tissue or the like) corresponding to a heat source, the optical fibers 511 also expand or compress together with the flexural element 401 as one piece body. As a result, the portion of the FBG sensor, i.e., the flexure detection element 301, also flexes. In such case, a flexure amount of the flexure detection element 301 can be calibrated into a temperature change of the first blade 201, using a calibration matrix.

According to the example depicted in FIG. 6, the optical fibers 511 are fixed to two positions near the tip and the base of the first blade 201. Accordingly, a flexure produced in the first blade 201 in a section between the two fixed points is detected by the flexure detection element 301 including the FBG sensor.

Note that FIG. 6 depicts only a part of the optical fiber constituting the FBG sensor used as the flexure detection element 301 (the part of the first blade 201 where the flexural element is formed) and does not depict other portions. It should be understood that an unillustrated opposite end of the optical fiber actually extends over the forceps rotation shaft 203 toward a detection unit and a signal processing unit (both not depicted).

The first blade 201 also flexes in at least one of the X, Y, or Z direction according to an acting force (e.g., external force applied at the time of contact with an operation target) as well as a temperature change. Accordingly, a detection signal generated by the flexure detection element including the FBG sensor can be measured as an acting force using a calibration matrix which performs calibration into an acting force on the first blade 201.

However, for separation between a flexure produced by an acting force on the first blade 201 and a flexure produced by a temperature change, a flexure detection element for detecting an acting force and a flexure detection element for detecting a temperature change need to be disposed separately from each other.

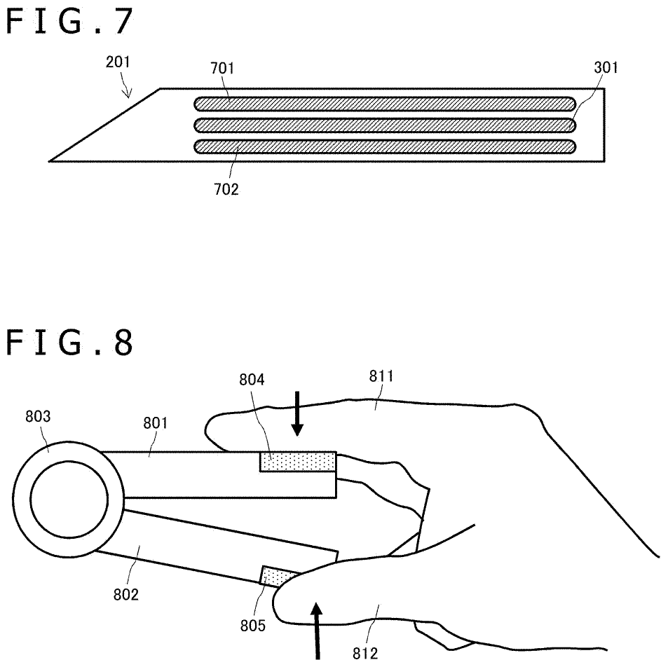

For example, as depicted in FIG. 7, a pair of flexure detection elements 701 and 702 for detecting an acting force may be provided on the first blade 201, independently of the flexure detection element 301 for detecting a temperature change. Each of the flexure detection elements 701 and 702 may include an FBG sensor similarly to the above configuration and may be embedded in and attached to a groove formed in the surface of the first blade 201 in the major axis direction. A flexure amount produced outside in the open-close direction of the first blade 201 is detectable using the flexure detection element 701 as one of the flexure detection elements, while a flexure amount generated inside in the open-close direction of the first blade 201 is detectable using the flexure detection element 702 as the other flexure detection element.

While the temperature sensor 51 is disposed only on the first blade 201 in the example described above with reference to FIGS. 3 to 6, note that the temperature sensor 51 which is an independent sensor may also be disposed on the second blade 202. In such case, a temperature can individually be measured for each of upper and lower surfaces of an operation target (a body tissue of the patient or the like) pinched by the first blade 201 and the second blade 202.

The temperature sense presentation unit 53 equipped on the master apparatus 10 side will next be described in detail.

The user remotely operates the support arm apparatus 210 and the end effector 200 on the slave apparatus 20 side, using the input unit 11 of the master apparatus 10. For example, the input unit 11 may include an input device such as a lever, a grip, a button, a jog dial, a tact switch, and a foot pedal switch (as described above). The present embodiment is characterized in that the temperature sense presentation unit 53 is provided on the input unit 11. More specifically, the temperature sense presentation unit 53 is disposed at a portion that comes into contact with a skin of a fingertip or the like of the user to give the user a temperature sense corresponding to a temperature of the end effector 200 (in other words, a temperature of a body tissue of the patient that is in contact with the end effector 200) measured by the temperature sensor 51. In this manner, a sense of touch with respect to the body tissue of the patient can effectively be reproduced.

FIG. 8 depicts such an example where the temperature sense presentation unit is disposed on a grip 800 used as the input unit 11 on the master apparatus 10 side. The grip 800 depicted in the figure has an open-close structure which pivotally supports a first grip member 801 and a second grip member 802 such that the respective grip members 801 and 802 are rotatable around a rotation shaft 803.

For example, the user can open and close the grip 800 by pressing the first grip 801 and the second grip member 802 using respectively a forefinger 811 and a thumb 812 of the right hand. In addition, on the slave apparatus 20 side, the drive unit 21 opens and closes the first blade 201 and the second blade 202 of the forceps 200 as the end effector according to a rotation angle of the grip 800 opened or closed by the forefinger 811 and the thumb 812 of the user. Accordingly, the user on the master apparatus 10 side can operate the grip 800 with a sense (or an analogy) of actually touching the body tissue of the patient using the forefinger 811 and the thumb 812.

Moreover, according to the present embodiment, a temperature actuator 804 and a temperature actuator 805 each functioning as the temperature sense presentation unit 53 are attached to the first grip member 801 and the second grip member 802 at respective portions in contact with the forefinger 811 and the thumb 812 of the user. Accordingly, the temperature of the body tissue of the patient can intuitively be presented to the user on the basis of the analogy (described above) between the first blade 201 and the second blade 202 and the forefinger and the thumb of the user by changing the temperatures to be presented by the temperature actuator 804 and the temperature actuator 805 according to the temperature of the end effector 200 (in other words, the temperature of the body tissue of the patient that is in contact with the end effector 200) measured by the temperature sensor 51. Moreover, a sense of touch with respect to the body tissue of the patient can effectively be reproduced by presenting a temperature sense to the user in addition to a force sense and a haptic sense.

Each of the temperature actuator 804 and the temperature actuator 805 includes a device capable of freely changing a temperature in at least a positive direction or a negative direction at least within a predetermined temperature range.

Note that the temperature actuator 804 and the temperature actuator 805 may be provided at a location where sensitivity of a fingertip is given priority over the analogy (described above) between the first blade 201 and the second blade 202 and the forefinger and the thumb of the user. Alternatively, the temperature actuator 804 and the temperature actuator 805 may be provided at a location defined in consideration of easy attachment to the grip 800.

Furthermore, according to the example depicted in FIG. 8, the temperature actuator 804 and the temperature actuator 805 are provided on the first grip member 801 and second grip member 802, respectively. However, a temperature can intuitively be presented to the user even if either the temperature actuator 804 or the temperature actuator 805 is provided alone.

In a case where the temperature sensor 51 is provided independently on each of the first blade 201 and the second blade 202 of the forceps as the end effector 200, note that a temperature difference between both side surfaces of a target (a body tissue of the patient or the like) treated by the end effector 200 can be presented in real time if the temperature actuator 804 and the temperature actuator 805 are provided on the first grip member 801 and the second grip member 802, respectively and the respective temperature actuators 804 and 805 are allowed to independently present the temperatures of the first blade 201 and the second blade 202, respectively.

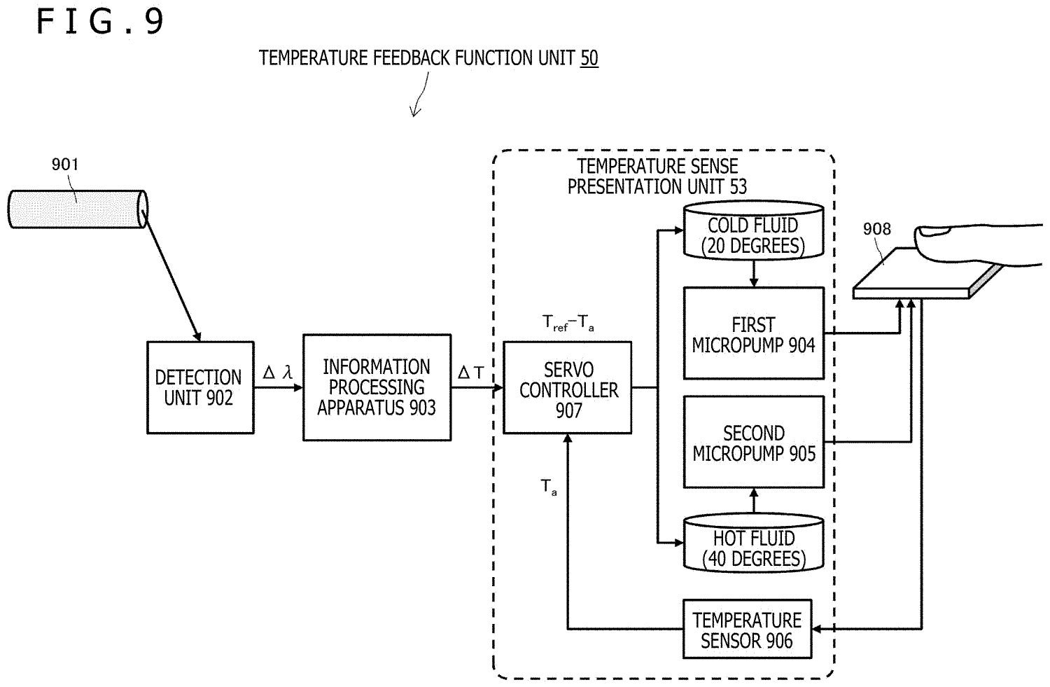

FIG. 9 depicts a specific configuration example of the temperature sense feedback function unit 50 incorporated in the master-slave system 1.

The FBG sensor 901 as the temperature sensor 51 attached to the end effector 200 generates a flexure Δε together with the end effector 200 as one piece body as a result of a temperature change ΔT. The FBG sensor 901 is a sensor including diffraction gratings (gratings) formed along the major axis of the optical fibers (as described above).

A detection unit (Interrogator) 902 receives detection light in a predetermined wavelength band (Bragg wavelength) from an end of the FBG sensor 901, and receives reflection light of the detection light to detect a change of intervals of diffraction gratings produced by the flexure Δε of the end effector 200 as a change of the wavelength Δλ of the reflection light. Here, it is assumed that the change of the wavelength Δλ of the FBG sensor 901 is substantially proportional to the flexure Δε produced in the end effector 200.

In a case where a flexure sensor other than the FBG sensor is employed as the temperature sensor 51, note that the detection unit 902 is assumed to detect the flexure Δε produced in the end effector 200 as a result of the temperature change ΔT, using this flexure sensor.

An information processing apparatus 903 receives input of the detection result Δλ obtained by the detection unit 902 and calibrates the wavelength change Δλ produced in the FBG sensor 901 into the temperature change ΔT in the end effector 200, using a calibration matrix determined beforehand. For example, assuming that a calibration gain is kₜₕ, the wavelength change Δλ of the FBG sensor 901 can be calibrated into the temperature change ΔT using the following Equation (1). In addition, in a case where a flexure sensor other than the FBG sensor is employed as the temperature sensor 51, the information processing apparatus 903 can calibrate the flexure Δε produced in the end effector 200 into the temperature change ΔT, using the following Equation (2) which has a calibration gain kₜₕ′.

[Math. 1]

ΔT=kₜₕ×Δλ (1)

[Math. 2]

ΔT=kₜₕ′×Δε (2)

Note that the information processing apparatus 903 is a personal computer (PC), for example. Moreover, the control system 30 (see FIG. 1) may have a function of the information processing apparatus 903 to perform a calibration calculation expressed in Equation (1) or (2) described above.

As described above, the temperature sense presentation unit 53 contacts the fingertip (or a skin of a different portion) of the user on the input unit 11 such as a grip. According to the example depicted in FIG. 9, the temperature sense presentation unit 53 includes a first micropump 904 which changes a temperature in a negative direction, a second micropump 905 which changes a temperature in a positive direction, a temperature sensor 906 which measures a temperature, and a servo controller 907.

The first micropump 904 receives supply of cold fluid (or cold gas) at 20° C., for example, and changes a temperature of a contact surface 908 in contact with the fingertip of the user, in the negative direction. On the other hand, the second micropump 905 receives supply of hot fluid (or hot gas) at 40° C., for example, and changes a temperature of the contact surface 908 in contact with the fingertip of the user, in the positive direction.

The servo controller 907 sets a target temperature T_{ref} of the temperature sense presentation unit 53 in response to input of a measurement result of the temperature change ΔT of the end effector 200 (in other words, a body tissue of the patient treated by the forceps as the end effector 200). Thereafter, the servo controller 907 gives instruction on a supply amount of cold fluid to the first micropump 904 and a supply amount of hot fluid to the second micropump 905 to achieve the target temperature T_{ref}.

In addition, the temperature sensor 906 measures an actual temperature Tₐ of the contact surface 908 in contact with the fingertip of the user and feeds back the measured temperature Tₐ to the servo controller 907. Thereafter, the servo controller 907 controls the supply amount of cold fluid to the first micropump 904 and the supply amount of hot fluid to the second micropump 905 on the basis of a difference between the target temperature T_{ref} and the actual temperature Tₐ, to perform servo control such that the temperature of the contact surface 908 in contact with the fingertip of the user approaches the target temperature T_{ref}.

A method for controlling a temperature of the temperature sense presentation unit 53 by the servo controller 907 will next be described in detail.

The servo controller 907 basically changes the temperature of the contact surface 908 in contact with the fingertip of the user, in harmony with a temperature of the end effector 200 (in other words, a temperature of a body tissue of a surgical site of the patient as an operation target) detected by the temperature sensor 51. More specifically, the servo controller 907 increases the temperature of the contact surface 908 in contact with the fingertip of the user according to an increase in a surface temperature of an operation target in contact with the end effector 200. Conversely, the servo controller 907 decreases the temperature of the contact surface 908 in contact with the fingertip of the user according to a decrease in the surface temperature of the operation target.

However, a ratio of the temperature change ΔT of the end effector 200 to a temperature change ΔT_{ref} of the target temperature T_{ref} of the temperature sense presentation unit 53 is not required to be 1 to 1. For example, if the servo controller 907 sets the ratio of the temperature change ΔT of the effector 200 to the temperature change ΔT_{ref} of the target temperature T_{ref} of the temperature sense presentation unit 53 to 1 to n (provided, on an assumption of n>1), the temperature presented by the temperature sense presentation unit 53 changes at a temperature change ΔT_{ref} (=n·ΔT) amplified to n times the temperature change ΔT of the end effector 200 (in other words, the body tissue of the patient corresponding to the operation target). In such manner, the user can sense a temperature change of the body tissue of the patient with substantially high sensitivity during a surgery.

Moreover, the servo controller 907 may set a ratio of the temperature change ΔT of the end effector 200 to the temperature change ΔT_{ref} of the target temperature T_{ref} of the temperature sense presentation unit 53 to n to 1 (provided, on an assumption of n>1). In such case, the temperature of the temperature sense presentation unit 53 changes at a temperature change ΔT_{ref} (=ΔT/n) damped to one nth of the temperature change of the end effector 200. For example, when the end effector 200 treats an object having an extremely high temperature or conversely an object having an extremely low sound, a temperature can be presented without invasion to the user operating the input unit 11 by compressing the temperature change ΔT to one nth.

Furthermore, it is known that human sense has a characteristic of being more sensitive to a change. Such characteristic is also applicable to a temperature sense. In other words, a human is capable of sensing a temperature change with sensitivity higher than that for a static temperature. Accordingly, the servo controller 907 may present a temperature to the user using the temperature sense presentation unit 53, with control over a speed or acceleration of the temperature change ΔT detected by the temperature sensor 51, to control sensitivity of the user for detecting a temperature.

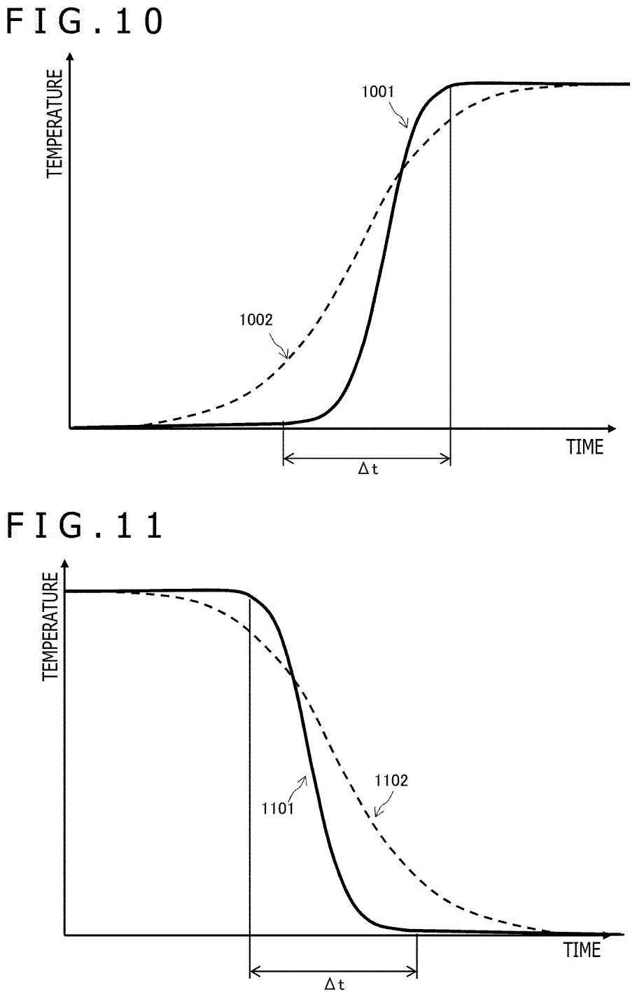

FIG. 10 depicts an example of a relationship between a temperature T detected by the temperature sensor 51 and the target temperature T_{ref} instructed to the temperature sense presentation unit 53 by the servo controller 907. Note that the temperature T detected by the temperature sensor 51 is indicated by a dotted line 1002 and that the target temperature T_{ref} is indicated by a solid line 1001, with a horizontal axis representing a time axis and a vertical axis representing a temperature axis. According to the example depicted in FIG. 10, the speed or the acceleration of the temperature change ΔT is controlled such that a rise time of the temperature change ΔT_{ref} of the target temperature T_{ref} is compressed within a human sensing limit. It is therefore expected that the user can detect the temperature change ΔT of the end effector 200 (or a body tissue of the patient treated by the end effector 200) with high sensitivity. It is preferable that a time Δt required for a rise of the presented temperature is set equal to or shorter than a human detection limit.

In addition, FIG. 11 depicts another example of the relationship between the temperature T detected by the temperature sensor 51 and the target temperature T_{ref} instructed to the temperature sense presentation unit 53 by the servo controller 907. Note that the temperature T detected by the temperature sensor 51 is indicated by a dotted line 1102 and that the target temperature T_{ref} is indicated by a solid line 1101, with a horizontal axis representing a time axis and a vertical axis representing a temperature axis. According to the example depicted in FIG. 11, the speed or the acceleration of the temperature change ΔT is controlled such that a fall time of the temperature change ΔT_{ref} of the target temperature T_{ref} is compressed within a human sensing limit. It is therefore expected that the user can detect the temperature change ΔT of the end effector 200 (or a body tissue of the patient treated by the end effector 200) with high sensitivity. It is preferable that a time Δt required for a fall of the presented temperature is set equal to or shorter than a human detection limit.

Moreover, a human sense may lower in sensitivity with habituation caused by exposure to the same environment for a certain period of time. Such characteristic is also applicable to a temperature sense. It is assumed that insensitivity to a temperature may be caused by exposure to the same temperature for a certain period of time. Accordingly, for preventing a decrease in the user's sensitivity in terms of the temperature sense, the servo controller 907 may reset the target temperature T_{ref} of the temperature sense presentation unit 53 (or the temperature presented to the user) to an initial temperature Tᵢₙᵢₜ and present a new temperature sense ΔT_{ref} after a certain period of time has elapsed from the time when the detected temperature T obtained by the temperature sensor 51 has ceased to change (or after a certain period of time has elapsed from the time when the temperature change ΔT has started to fall under a threshold ΔTₜₕ), or after a certain period of time has elapsed from the time when the target temperature T_{ref was} has last been changed.

A determination process performed by the determination unit 60 on the basis of a measured temperature will next be described in detail.

The determination unit 60 determines a state of an affected site of the patient indicated by a temperature measured by the temperature sensor 51, with reference to the data table 61 which describes a correlation between a temperature and a state of a surgical site of the patient. For example, when blood comes into contact with the temperature sensor 51, the determination unit 60 determines that a surgical site of the patient corresponding to an operation target is bleeding, on the basis of a temperature difference between an environmental temperature and the blood. In addition, when the temperature sensor 51 touches an object that is present at an invisible place, the determination unit 60 determines which tissue is in contact (e.g., which of fat and a blood vessel), on the basis of a temperature difference.

Moreover, the determination unit 60 determines whether or not an operation target (a body tissue of the patient) in contact with the end effector is a normal tissue on the basis of a combination of a temperature detected by the temperature sensor 51 and information indicating an external force (i.e., force sense) that is acting on the end effector and is detected by the state detection unit 22, with reference to the data table 61 which describes a correlation between a temperature and a force sense and a state of a body tissue of the patient.

In a case where the end effector 200 includes a device which has two or more contact portions in contact with an operation target, such as the forceps depicted in FIG. 3 or other figures, the temperature sensor 51 may be provided on one of the contact portions, and further, the temperature input unit 54 may be provided on another contact portion.

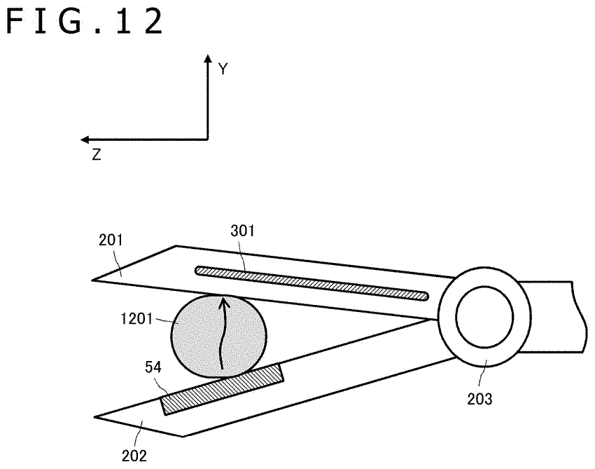

FIG. 12 depicts a configuration example of the end effector (forceps) 200 which includes the flexure detection element 301 as a temperature sensor provided on the first blade 201 and the temperature input unit 54 provided on the second blade 202.

The flexure detection element 301 provided on the first blade 201 includes an FBG sensor, for example. The configuration of the first blade 201 has already been described with reference to FIGS. 3 to 6, and therefore, detailed description of this configuration is omitted here.

On the other hand, the temperature input unit 54 provided on the second blade 202 includes a temperature actuator, such as a heating element, capable of freely changing a temperature at least within a predetermined temperature range.

As depicted in FIG. 12, when a temperature is input from the temperature input unit 54 via one side surface of an operation target 1202 (for example, the operation target 1201 is heated) in a state where the operation target is pinched between the first blade 201 and the second blade 202, the input temperature is transmitted to the opposite side surface via the operation target 1201 and conducted to the first blade 201. As a result, the first blade 201 thermally expands, and a flexure corresponding to a temperature change is detected by the flexure detection element 301. Thereafter, the flexure produced in the first blade 201 is calibrated into a temperature change, using a predetermined calibration matrix.

The determination unit 60 is capable of determining a type or a characteristic of the operation target 1201 on the basis of a relationship between a temperature input to the one side surface of the operation target 1201 from the temperature input unit 54 and a temperature of the opposite side surface of the operation target 1201 measured by the temperature sensor 51. For example, a delay time upon conduction of the input temperature in the operation target 1201 and a damping manner of the input temperature according to conduction are characterized in correspondence with the type or the characteristic of the operation target 1201. Accordingly, the determination unit 60 can perform a determination process for determining the type or the characteristic of the operation target 1201 with reference to the data table 61 which describes a correspondence between a transmission function of an input/output temperature characteristic and the type or the characteristic of the operation target (a tissue of a surgical site or the like). Moreover, the determination unit 60 may include a learning machine created by machine learning of teacher data indicating a temperature and a state of a surgical site of the patient, with use of a neural network or the like.

Basically, the user remotely operating the slave apparatus 10 from the master apparatus 10 side is notified of a determination result obtained by the determination unit 60. For example, a determination result obtained by the determination unit 60 is displayed on the screen of the display 53-2 of the temperature sense presentation unit 53. For example, a determination result may be superimposed on an image of a surgical site of the patient captured by an endoscope (an image of a bleeding portion, a figure indicating a portion corresponding to a surgery target, or the like is superimposed, for example). Needless to say, a determination result obtained by the determination unit 60 may be displayed on the screen as character information such as a comment instead of an image.

Moreover, the user may be notified of the determination result obtained by the determination unit 60 not as a notification on the screen but by audio output such as a voice guidance. Furthermore, the determination result obtained by the determination unit 60 may be superimposed on a force sense presented by the force sense presentation unit 12, a haptic sense presented by the vibration presentation unit 45, and a temperature sense presented by the temperature sense presentation unit 53 (for example, the user is notified of an emergency through a force sense, a haptic sense, or a temperature sense at the time it has been determined that bleeding from an affected site has occurred).

According to the system configuration example depicted in FIG. 1, the determination unit 60 is disposed on the master apparatus 10 side. In a modification, the determination unit 60 may be provided not on the master apparatus 10 but on the slave apparatus 20 side and may transmit a determination result obtained by the determination unit 60 to the master apparatus 10 side.

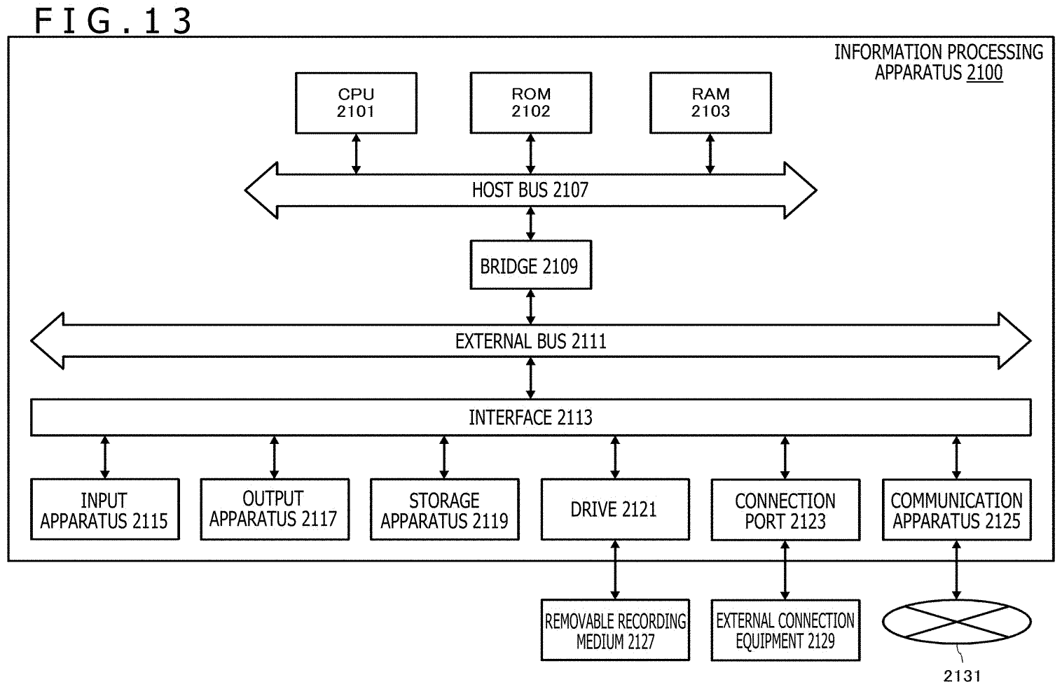

FIG. 13 depicts a hardware configuration of an information processing apparatus 2100 capable of operating as the control system 30 of the master-slave system 1 depicted in FIG. 1. The information processing apparatus 903 in the temperature sense feedback function unit 50 depicted in FIG. 9 may also be used as the information processing apparatus 2100 depicted in the figure.

The information processing apparatus 2100 depicted in the figure chiefly includes a CPU 2101, a ROM (Read Only Memory) 2103, and a RAM (Random Access Memory) 2105, and further includes a host bus 2107, a bridge 2109, an external bus 2111, an interface 2113, an input apparatus 2115, an output apparatus 2117, a storage apparatus 2119, a drive 2121, a connection port 2123, and a communication apparatus 2125.

The CPU 2101 functions as a calculation processor and a controller and controls an entire operation or a part of the entire operation in the information processing apparatus 2100 under various programs recorded in the ROM 2103, the RAM 2105, the storage apparatus 2119, or a removable recording medium 2127. The ROM 2103 stores programs, calculation parameters, and the like used by the CPU 2101, in a non-volatile manner. The RAM 2105 temporarily stores programs used by the CPU 2101, parameters that change appropriately upon execution of the programs, and the like. These units are connected to each other via the host bus 2107 including an internal bus such as a CPU bus. Note that the function of the control system 30 in the robot system 1 depicted in FIG. 1 may be implemented under a predetermined program executed by the CPU 2101, for example.

The host bus 2107 is connected to the external bus 2111 such as a PCI (Peripheral Component Interconnect) bus via the bridge 2109. In addition, the input apparatus 2115, the output apparatus 2117, the storage apparatus 2119, the drive 2121, the connection port 2123, and the communication apparatus 2125 are connected to the external bus 2111 via the interface 2113.

For example, the input apparatus 2115 includes an operation device operated by the user, such as a mouse, a keyboard, a touch panel, a button, a switch, a lever, and a pedal. Moreover, for example, the input apparatus 2115 may be a remote controller (what is generally called a remote) which uses infrared light or other radio waves or may be external connection equipment 2129 such as a cellular phone, a smartphone, and a PDA (Personal Digital Assistant) corresponding to an operation of the information processing apparatus 2100. Furthermore, for example, the input apparatus 2115 may include an input control circuit which generates an input signal on the basis of information input from the user using the operation device described above and outputs the generated input signal to the CPU 2101. The user of the information processing apparatus 2100 is capable of inputting various types of data to the information processing apparatus 2100 and giving an instruction on a processing operation by operating the input apparatus 2115.

The output apparatus 2117 includes an apparatus capable of notifying the user of acquired information in a visual or auditory manner. Examples of such an apparatus include a display apparatus such as a CRT display apparatus, a liquid crystal display apparatus, a plasma display apparatus, an EL display apparatus, and a lamp, an audio output apparatus such as a speaker and a headphone, and a printer apparatus.

For example, the output apparatus 2117 outputs a result obtained by various processes performed by the information processing apparatus 2100. More specifically, the display apparatus displays a result obtained by various processes performed by the information processing apparatus 2100 in the form of text or an image. On the other hand, the audio output apparatus converts an audio signal including reproduced audio data and acoustic data into an analog signal and outputs sounds. Note that a monitor 260 included in the master apparatus 10 may be implemented by the output apparatus 2117, for example. In addition, the display 53-2 of the temperature sense presentation unit 53 may also be used as the output apparatus 2117.

The storage apparatus 2119 is an apparatus for data storage constituted as an example of a storage unit of the information processing apparatus 2100. Examples of the storage apparatus 2119 include a magnetic storage device such as an HDD (Hard Disk Drive), a semiconductor storage device, an optical storage device, and a magneto-optical storage device. The storage apparatus 2119 stores programs executed by the CPU 2101, various kinds of data, and the like.

The drive 2121 is a reader-writer for recording medium and is built in or externally attached to the information processing apparatus 2100. The drive 2121 reads out information recorded in the attached removable recording medium 2127 such as a magnetic disk, an optical disk, a magneto-optical disk, and a semiconductor memory and outputs the information to the RAM 2105 and the like. The drive 2121 is also capable of writing a record into the attached removable recording medium 2127 such as a magnetic disk, an optical disk, a magneto-optical disk, and a semiconductor memory. For example, the removable recording medium 2127 is a DVD medium, an HD-DVD medium, a Blu-ray (registered trademark) medium, or the like. Moreover, the removable recording medium 2127 may be a Compact Flash (registered trademark) (CF: Compact Flash), a flash memory, an SD memory card (Secure Digital memory card), or the like. Furthermore, for example, the removable recording medium 2127 may be an IC (Integrated Circuit) card equipped with a contactless IC chip, electronic equipment, or the like.

The connection port 2123 is a port for directly connecting to the information processing apparatus 2100. Examples of the connection port 2123 include an USB (Universal Serial Bus) port, an IEEE1394 port, an SCSI (Small Computer System Interface) port, and the like. Other examples of the connection port 2123 include an RS-232C port, an optical audio terminal, HDMI (registered trademark) (High-Definition Multimedia Interface) port, and the like. By connecting the external connection equipment 2129 to the connection port 2123, the information processing apparatus 2100 directly acquires various kinds of data from the external connection equipment 2129 or provides various kinds of data to the external connection equipment 2129.

For example, the communication apparatus 2125 is a communication interface which includes a communication device for connecting to a communication network (network) 2131, and the like. For example, the communication apparatus 2125 is a communication card for a wired or wireless LAN (Local Area Network), Bluetooth (registered trademark), or a WUSB (Wireless USB). In addition, the communication apparatus 2125 may be a router for optical communication, a router for ADSL (Asymmetric Digital Subscriber Line), a modem for various types of communication, or the like. For example, the communication apparatus 2125 is capable of transmitting and receiving a transmission signal to and from the Internet or other communication equipment under a predetermined protocol such as TCP/IP. In addition, the communication network 2131 connected to the communication apparatus 2125 may include a network connected by wire or without wire and the like and may be the Internet, a home LAN, infrared communication, radio wave communication, or satellite communication, for example.

The example of the hardware configuration of the information processing apparatus 2100 capable of implementing the function of the control system 30 of the master-slave system 1 according to the present embodiment has been described above. The respective constituent elements described above may be provided by using general-purpose components or may be provided by hardware specialized to function as the respective constituent elements. Accordingly, the hardware configuration to be used may appropriately be modified according to a technical level for each occasion at which the present embodiment is carried out. While not depicted in FIG. 13, it is assumed that various configurations corresponding to the information processing apparatus 2100 constituting the control system 30 according to the present embodiment are naturally provided.

Note that a computer program for implementing the respective functions of the information processing apparatus 2100 constituting the control system 30 according to the present embodiment described above can be created and installed in a personal computer or the like. Moreover, a recording medium storing such a computer program and readable by a computer can be provided. For example, the recording medium is a magnetic disk, an optical disk, a magneto-optical disk, a flash memory, or the like. Furthermore, the computer program described above may be distributed via a network, for example, without using the recording medium. In addition, the number of computers executing the corresponding computer program is not particularly limited. For example, the corresponding computer program may be executed by plural computers (e.g., plural servers) in cooperation with each other. Note that a single computer or plural computers operating in cooperation with each other is also referred to as a “computer system.”

According to the master-slave system to which the technology disclosed in the present description is applied, a surface temperature of an operation target in contact with an end effector on the slave apparatus side can be measured and presented to a user remotely operating the slave apparatus on the master apparatus side, in real time. In such manner, the user is allowed to handle the tip of the slave apparatus “as if the tip is his or her hand.” Moreover, invasion can be reduced more than an endoscopic surgery called HALS (Hand-assisted Laparoscopic Surgery) where a surgeon performs a surgery while directly touching a tissue through a port through which one hand is inserted, in addition to the use of a surgical tool such as forceps and an endoscope.

There are various methods for presenting the surface temperature of the operation target on the master apparatus side. For example, the master apparatus may present a temperature sense corresponding to a measured surface temperature to the user or may present information associated with the surface temperature using a color or a numerical value, for example, and display the information on a screen. Moreover, the master apparatus may present a temperature sense while changing a temperature in harmony with the measured surface temperature. At this time, the temperature change of the surface temperature may be amplified and presented to allow the user to easily detect the temperature change of the operation target.

In a case where the master-slave system is applied to a medical field, a space between the hands is needed to identify the body tissue or determine any contact with a body tissue. According to the master-slave system to which the technology disclosed in the present description is applied, a state of an affected site of a patient can be determined on the basis of a difference between a temperature detected from a body tissue or blood under treatment and an environmental temperature. Moreover, according to the master-slave system to which the technology disclosed in the present description is applied, a type or a characteristic of an operation target can be determined on the basis of a transmission function of an input or output temperature characteristic of an operation target.

Furthermore, according to the master-slave system to which the technology disclosed in the present description is applied, a temperature sense can be presented to a user of the master apparatus remotely operating the slave apparatus, in addition to a force sense and a vibrational haptic sense. Accordingly, the user can easily obtain a sense of touch during an endoscopic surgery or the like through the master apparatus. By presenting a real-time temperature sense to the user, performance achieved by tissue determination and contact determination improves, and further, safety achieved by contact detection and bleeding detection can increase. As a result, contribution to expansion of abilities of the user, such as precise operation and reduction of invasion, and efficient performance of surgeries are achievable while transmission functions of the slave apparatus and the master apparatus are reduced to one function.

### INDUSTRIAL APPLICABILITY

The technology disclosed in the present description has been described in detail above with reference to the specific embodiment. However, it is obvious that those skilled in the art are allowed to make corrections or substitutions of this embodiment without departing from the subject matter of the technology disclosed in the present description.

While an embodiment in which the technology disclosed in the present description is applied to a medical robot apparatus for an endoscopic surgery or the like has chiefly been described in the present description, the technology disclosed in the present description is also applicable to robot apparatuses used in various fields other than the medical field. Moreover, the technology disclosed in the present description is also applicable to robot apparatuses of various types other than the master-slave type.

Furthermore, the technology disclosed in the present description may be applied to an entertainment field such as virtual reality (VR) and present a real-time temperature sense to provide a user experience in a more realistic manner.

In short, the technology disclosed in the present description has been described by way of example, and therefore, the contents described in the present description should not be interpreted in a limited manner. The appended claims should be taken into consideration for determining the subject matter of the technology disclosed in the present description.

Note that the technology disclosed in the present description may also have the following configurations.

(1)

A master-slave system including:

a slave apparatus that includes a temperature acquisition unit that acquires a temperature; and

a master apparatus that includes a presentation unit that presents the acquired temperature.

(2)

The master-slave system according to (1) described above, in which

the temperature acquisition unit includes a temperature sensor attached to an end effector of the slave apparatus.

(3)

The master-slave system according to (1) or (2) described above, in which

the presentation unit presents a temperature sense corresponding to the temperature acquired by the temperature acquisition unit.

(4)

The master-slave system according to any one of (1) to (3) described above, in which

the presentation unit includes a display unit that displays information associated with the temperature acquired by the temperature acquisition unit.

(5)

The master-slave system according to (4) described above, in which

the display unit displays a numerical value or a color corresponding to a temperature.

(6)

The master-slave system according to any one of (1) to (5) described above, further including:

a determination unit that determines a state of an operation target treated by the end effector, on the basis of the temperature acquired by the temperature acquisition unit.

(7)

The master-slave system according to (6) described above, further including:

a force sensor that detects an external force acting on the end effector, in which

the determination unit determines the state of the operation target on the basis of a combination of a force sense obtained by the force sensor and information indicating the temperature acquired by the temperature acquisition unit.

(8)

The master-slave system according to (6) or (7) described above, in which

the presentation unit further presents information associated with a determination result obtained by the determination unit.

(9)

The master-slave system according to any one of (1) to (8) described above, in which

the master apparatus includes an operation unit used to operate the slave apparatus, and

the presentation unit presents a temperature sense based on the acquired temperature, by the operation unit.

(10)

The master-slave system according to (9) described above, in which

the presentation unit includes a temperature change unit that is provided on the operation unit and produces a temperature change on the basis of the acquired temperature.

(11)

The master-slave system according to (10) described above, in which

the temperature change unit produces the temperature change in harmony with the acquired temperature.

(12)

The master-slave system according to (11) described above, in which

the temperature change unit produces a temperature change of the temperature acquired by the temperature acquisition unit while amplifying or damping the temperature change.

(13)

The master-slave system according to (11) or (12) described above, in which

the temperature change unit produces a temperature change of the temperature acquired by the temperature acquisition unit while controlling a speed or

acceleration of the temperature change.

(14)

The master-slave system according to any one of (11) to (13) described above, in which

the temperature change unit performs a reset to an initial temperature after a certain period of time has elapsed from the time when the temperature has ceased to change.

(15)

The master-slave system according to any one of (10) to (14) described above, in which

the temperature change unit includes either a micropump or a Peltier element.

(16)

The master-slave system according to any one of (1) to (15) described above, in which

the slave apparatus includes an end effector that includes two or more contact portions in contact with an operation target,

the temperature acquisition unit acquires a temperature of one of the contact portions, and

a temperature input unit that inputs a temperature into the operation target is provided on another contact portion.

(17)

The master-slave system according to (16) described above, further including:

a second determination unit that determines a type or a characteristic of the operation target on the basis of an input temperature input by the temperature input unit and the temperature acquired by the temperature acquisition unit.

(18)

The master-slave system according to (2) described above, in which

the temperature sensor includes a flexure sensor capable of detecting a flexure produced in the end effector due to a temperature change.

(19)

The master-slave system according to (18) described above, in which

the flexure sensor includes an FBG (Fiber Bragg Grating) sensor.

(20)

A control method of a master-slave system, the control method including:

a temperature acquisition step that causes a slave apparatus to acquire a temperature; and

a presentation step that causes a master apparatus to present the temperature acquired by the slave apparatus.

### CITATION LIST

### Patent Literature

### [PTL 1]

Japanese Patent Laid-open No. 2016-214715

### [PTL 2]

Japanese Patent Publication 4-45310

### REFERENCE SIGNS LIST

1 Master-slave system

10 Master apparatus, 11 Input unit, 12 Force presentation unit

20 Slave apparatus, 21 Drive unit, 22 State detection unit

30 Control system

40 Vibration feedback function unit

41 First vibration sensor, 42 Second vibration sensor

43 Vibration transmission unit, 44 First vibration presentation unit, 45 Second vibration presentation unit

50 Temperature sense feedback function unit

51 Temperature sensor, 52 Temperature transmission unit, 53 Temperature sense presentation unit

53-1 Temperature actuator, 53-2 Display

54 Temperature input unit

60 Determination unit, 61 Data table

200 End effector (forceps)

201 First blade, 202 Second blade

203 Forceps rotation shaft

210 Support arm apparatus

301 Flexure detection element, 401 Flexural element

501 Groove, 511 Optical fiber

800 Grip

801 First grip member, 802 Second grip member

803 Rotation shaft, 804, 805 Temperature actuator

901 FBG sensor, 902 Detection unit, 903 Information processing apparatus

904 First micropump, 905 Second micropump

906 Temperature sensor, 907 Servo controller

2100 Information processing apparatus, 2101 CPU, 2103 ROM

2105 RAM, 2107 Host bus, 2109 Bridge

2111 External bus, 2113 Interface, 2115 Input apparatus

2117 Output apparatus, 2119 Storage apparatus, 2121 Drive

2123 Connection port, 2125 Communication apparatus

1. A master-slave system comprising:
a slave apparatus that includes a temperature acquisition unit that acquires a temperature; and
a master apparatus that includes a presentation unit that presents the acquired temperature.

2. The master-slave system according to claim 1, wherein
the temperature acquisition unit includes a temperature sensor attached to an end effector of the slave apparatus.

3. The master-slave system according to claim 1, wherein
the presentation unit presents a temperature sense corresponding to the temperature acquired by the temperature acquisition unit.

4. The master-slave system according to claim 1, wherein
the presentation unit includes a display unit that displays information associated with the temperature acquired by the temperature acquisition unit.

5. The master-slave system according to claim 4, wherein
the display unit displays a numerical value or a color corresponding to a temperature.

6. The master-slave system according to claim 1, further comprising:
a determination unit that determines a state of an operation target treated by the end effector on a basis of the temperature acquired by the temperature acquisition unit.

7. The master-slave system according to claim 6, further comprising:
a force sensor that detects an external force acting on the end effector, wherein
the determination unit determines the state of the operation target on a basis of a combination of a force sense obtained by the force sensor and information indicating the temperature acquired by the temperature acquisition unit.

8. The master-slave system according to claim 6, wherein
the presentation unit further presents information associated with a determination result obtained by the determination unit.

9. The master-slave system according to claim 1, wherein
the master apparatus includes an operation unit used to operate the slave apparatus, and
the presentation unit presents a temperature sense based on the acquired temperature, by the operation unit.

10. The master-slave system according to claim 9, wherein
the presentation unit includes a temperature change unit that is provided on the operation unit and produces a temperature change on a basis of the acquired temperature.

11. The master-slave system according to claim 10, wherein
the temperature change unit produces the temperature change in harmony with the acquired temperature.

12. The master-slave system according to claim 11, wherein
the temperature change unit produces a temperature change of the temperature acquired by the temperature acquisition unit while amplifying or damping the temperature change.

13. The master-slave system according to claim 11, wherein
the temperature change unit produces a temperature change of the temperature acquired by the temperature acquisition unit while controlling a speed or acceleration of the temperature change.

14. The master-slave system according to claim 11, wherein
the temperature change unit performs a reset to an initial temperature after a certain period of time has elapsed from a time when the temperature has ceased to change.

15. The master-slave system according to claim 10, wherein
the temperature change unit includes either a micropump or a Peltier element.

16. The master-slave system according to claim 1, wherein
the slave apparatus includes an end effector that includes two or more contact portions in contact with an operation target,
the temperature acquisition unit acquires a temperature of one of the contact portions, and a temperature input unit that inputs a temperature into the operation target is provided on another contact portion.

17. The master-slave system according to claim 16, further comprising:
a second determination unit that determines a type or a characteristic of the operation target on a basis of an input temperature input by the temperature input unit and the temperature acquired by the temperature acquisition unit.

18. The master-slave system according to claim 2, wherein
the temperature sensor includes a flexure sensor capable of detecting a flexure produced in the end effector due to a temperature change.

19. The master-slave system according to claim 18, wherein
the flexure sensor includes an FBG (Fiber Bragg Grating) sensor.

20. A control method of a master-slave system, the control method comprising:
a temperature acquisition step that causes a slave apparatus to acquire a temperature; and
a presentation step that causes a master apparatus to present the temperature acquired by the slave apparatus.
